(19) United States
(12) Patent Application Publication
(10) Pub. No.: US 2016/0331245 A1
(43) Pub. Date: Nov. 17, 2016

(54) SEMI-AUTOMATIC SPHYGMOMANOMETER SYSTEM

(71) Applicant: KOVEN TECHNOLOGY, Creve Coeur, MO (US)

(72) Inventor: Frank Herndon, Creve Coeur, MO (US)

(21) Appl. No.: 15/219,885

(22) Filed: Jul. 26, 2016

Related U.S. Application Data
(63) Continuation-in-part of application No. 14/483,913, filed on Sep. 11, 2014.

(51) Int. Cl. A61B 5/022; A61B 5/0235

(52) U.S. Cl. CPC A61B 5/022; A61B 5/02225; A61B 5/0235

(57) ABSTRACT

Various embodiments of a semi-automatic sphygmomanometer apparatus that automatically determines whether a vascular cuff is configured with a large volume or a small volume are disclosed. The semi-automatic sphygmomanometer apparatus includes a pump in fluid flow communication with the vascular cuff and a processor in operative communication with a pressure detector for detecting a first plurality of cuff pressures over a first plurality of times and a second plurality of cuff pressure over a second plurality of times. At any stage during the inflation cycle including partially inflated vascular cuffs prior to re-inflation of the vascular cuff, the processor calculates a first average cuff pressure based on the first plurality of cuff pressures and a second average cuff pressure based on the second plurality of cuff pressures and then subtracting the first average cuff pressure from the second average cuff pressure to determine a slope value which is compared against a boundary value to determine whether the vascular cuff is configured with a large volume or a small volume.

### CROSS REFERENCE TO RELATED APPLICATIONS

This application is a continuation-in-part that claims benefit to U.S. non-provisional application Ser. No. 14/483,913 filed on Sep. 11, 2014, which claims benefit to U.S. provisional patent application Ser. No. 61/881,362 filed on Sep. 23, 2013, which are herein incorporated by reference in their entirety.

### FIELD

This document relates to a sphygmomanometer, and in particular, to a sphygmomanometer that automatically determines the size of a vascular cuff attached to the sphygmomanometer prior to re-inflation.

### BACKGROUND

A sphygmomanometer is a device used with a vascular cuff and stethoscope for measuring blood pressure in an artery. Typically, a manual sphygmomanometer is connected to a vascular cuff that is wrapped around an arm (or other location as required) of an individual and then inflated to constrict the flow of blood. The vascular cuff is connected to hollow tubing that communicates with a flexible bulb that is manually pumped by the practitioner to cause inflation of the vascular cuff. In addition, a valve and indicator gauge arrangement is interposed between the hollow tubing and the flexible bulb to provide a visual indication of pressure being applied by the vascular cuff and to control the flow of air pressure to the vascular cuff. A stethoscope or Doppler probe is applied over the individual's brachial pulse, which is located on the inside of the individual's upper arm near the elbow (or other location as required), and the valve is then tightened prior to repeatedly pumping the flexible bulb until the vascular cuff reaches a certain pressure, for example 200 mm Hg being shown on the indicator gauge. The vascular cuff is then slowly deflated by loosening the valve or another embodiment, such as an air release trigger. While watching the air pressure fall, the practitioner uses the stethoscope or Doppler probe to listen for when the first audible sound of a heartbeat or pulse of the individual is first heard which provides systolic blood pressure. Once the systolic blood pressure is determined, the valve may be completely opened and the remaining air let out of the vascular cuff. When the vascular cuff is completely bled, the sphygmomanometer can automatically detect the size of the vascular cuff attached to the individual; however, in many instances the vascular cuff may remain partially inflated rather than completely bled of air. In such instances where the vascular cuff remains partially inflated, it is difficult for the sphygmomanometer to automatically determine the size of the vascular cuff, thereby requiring manual intervention. As such, there is a need for a sphygmomanometer that automatically determines the size of the attached vascular cuff that is either completely bled of air or partially inflated prior to re-inflation of the vascular cuff.

### BRIEF DESCRIPTION OF THE DRAWINGS

FIG. 1 is a perspective view of a semi-automatic sphygmomanometer system showing the sphygmomanometer apparatus, a vascular cuff, and hollow tubing, according to aspects of the present disclosure;

FIG. 2 is a simplified block diagram of the semi-automatic sphygmomanometer system of FIG. 1, according to aspects of the present disclosure;

FIG. 3 is a perspective view of the sphygmomanometer apparatus of FIG. 1, according to aspects of the present disclosure;

FIG. 4 is a front view of the sphygmomanometer apparatus of FIG. 1, according to aspects of the present disclosure;

FIG. 5 is a perspective view of the sphygmomanometer apparatus and related ancillary equipment, according to aspects of the present disclosure;

FIG. 6 is a cross-sectional view of the sphygmomanometer apparatus taken along line 6-6 of FIG. 5, according to aspects of the present disclosure;

FIG. 7 is a flow chart illustrating one method for determining whether the vascular cuff is a large-sized vascular cuff or a small-sized vascular cuff, according to aspects of the present disclosure; and

FIG. 8 is a graph illustrating the determination of a boundary value for distinguishing large-sized vascular cuffs and small-sized vascular cuffs, according to aspects of the present disclosure.

Corresponding reference characters indicate corresponding elements among the view of the drawings. The headings used in the figures do not limit the scope of the claims.

### DETAILED DESCRIPTION

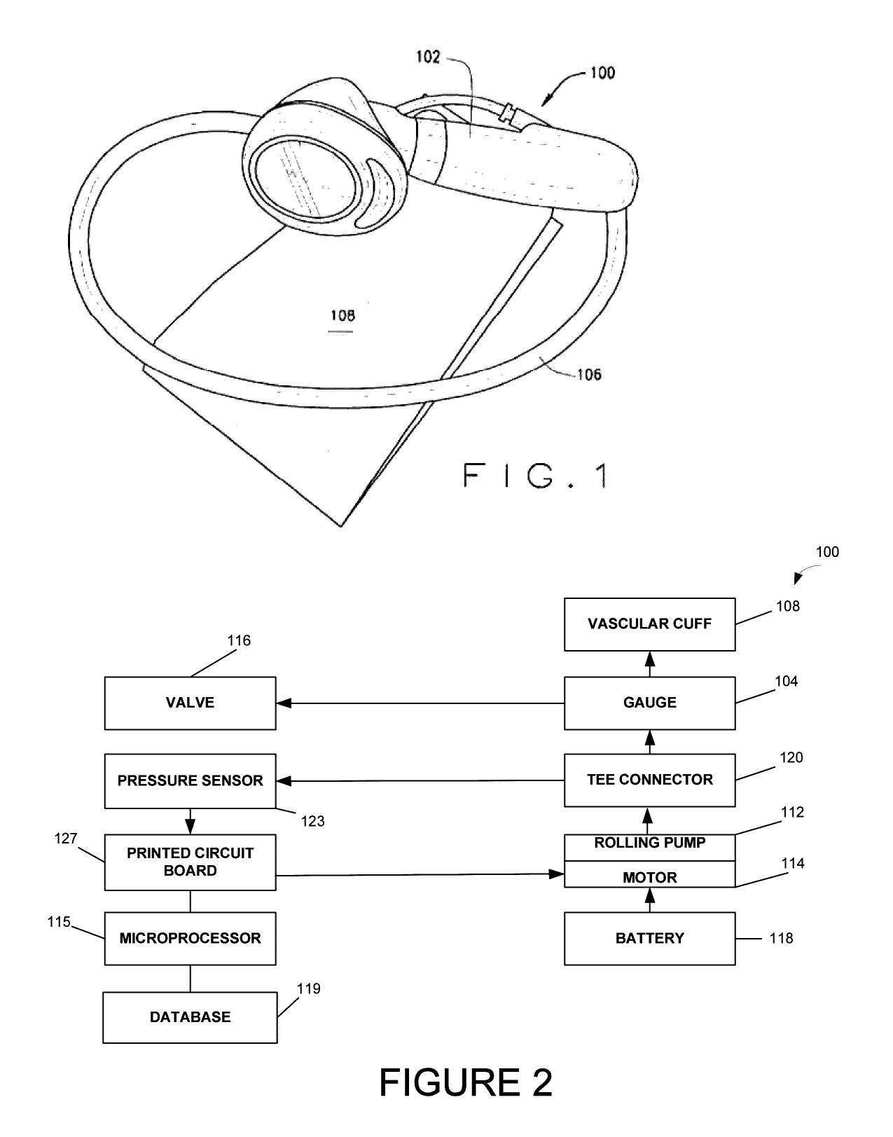

Various embodiments of a semi-automatic sphygmomanometer system operable for semi-automatic inflation of a vascular cuff including manually terminating inflation of the vascular cuff by a practitioner as an individual's blood pressure is being monitored are disclosed. In one aspect, the sphygmomanometer system provides a method for automatically detecting the size of a vascular cuff attached to an individual at any stage during the inflation cycle including partially inflated vascular cuffs prior to re-inflation of the vascular cuff. Unlike blood pressure meters that distinguish different sized cuffs in the non-inflated (e.g., 0 mmHg) state, the auscultator method of detecting blood pressure may occasionally require distinguishing different sized cuffs in partially inflated states. Comparing initial inflation slopes to known library of vascular cuffs of different sizes is effective only when inflating from the non-inflated state (0 mmHg); however, it is not effective in determining appropriate vascular cuff sizes during re-inflation of a vascular cuff at already elevated pressure levels, such as 200 mmHg. In another aspect, the sphygmomanometer system includes a processor that applies an algorithmic process to determine vascular cuff size to appropriately detect and select the correct vascular cuff size at any pressure being applied to the vascular cuff. Referring to the drawings, an embodiment of a semi-automatic sphygmomanometer system is illustrated and generally indicated as 100 in FIGS. 1-8. As shown in FIGS. 1 and 2, in some embodiments a semi-automatic sphygmomanometer system 100 may include a sphygmomanometer apparatus 102 connected to a vascular cuff 108 through hollow tubing 106 in which sphygmomanometer apparatus 102 allows for semi-automatic inflation of the vascular cuff 108 and manual termination of inflation of the vascular cuff 108 by the practitioner.

As further shown in FIG. 2, in some embodiments the semi-automatic sphygmomanometer system 100 may include a motor 114 in operative connection to a rolling pump 112 for generating air flow through a tee connector 120. The tee connector 120 receives pressurized air from the rolling pump 112 and splits the pressurize air into two separate air pathways—a first pathway that supplies the pressurized air to an indicator gauge 104 for indicating the amount of air pressure, a valve 116, and vascular cuff 108; and a second pathway that supplies the pressurized air to a pressure sensor 123.

The pressure sensor 123 is in operative communication with a printed circuit board 127 having a microprocessor 115 disposed within the sphygmomanometer apparatus 102 and in operative communication with a database 119. The microprocessor 115 may be any type of processor suitable for placement on the printed circuit board 127 and is operable to receive air pressure data from the pressure sensor 123 as the vascular cuff 108 is being inflated with pressurized air from the rolling pump 112. In one embodiment, the microprocessor 115 may automatically determine the size of the vascular cuff 108 connected to the sphygmomanometer apparatus 102. In particular, in one aspect the microprocessor 115 can determine whether the vascular cuff 108 is either a small-sized vascular cuff or a large-sized vascular cuff based on the data received from the pressure sensor 123 and processed by the microprocessor 115 regardless of whether the vascular cuff 108 is in a non-inflated state or a partially inflated state based on the method of operation discussed below. In some embodiments, the small-sized vascular cuff may have a size of about 10 cm³ and the large sized vascular cuff may have a size of about 300 cm³.

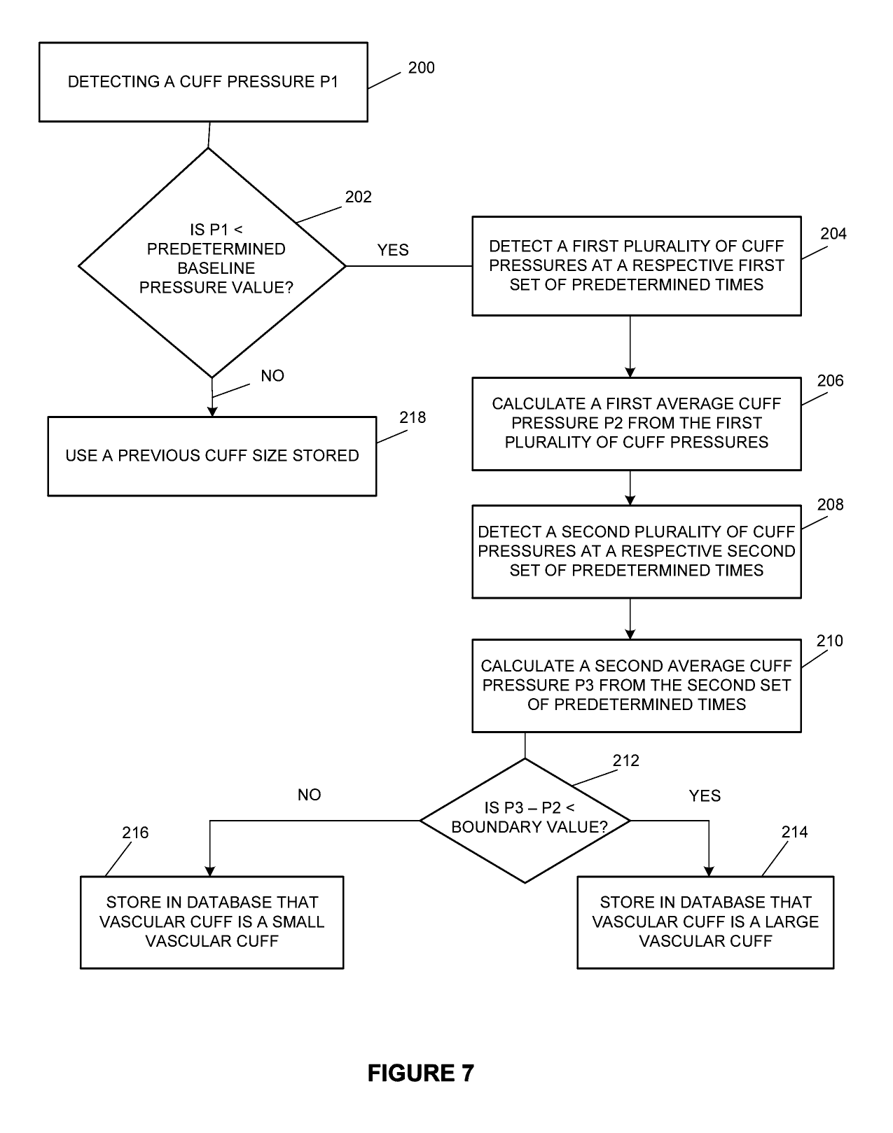

Referring to FIG. 7, one method for automatically determining whether the vascular cuff 108 is either a large-sized vascular cuff or a small-sized vascular cuff is illustrated. At block 200, the pressure sensor 123 detects a cuff pressure P1 from the vascular cuff 108 at a time T1 (time in millisecond). At decision point 202, the microprocessor 115 determines whether the cuff pressure P1 is less than a predetermined baseline pressure value, for example 70 mmHg, or whether the cuff pressure P1 is greater than the predetermined baseline pressure value. In some embodiments, the predetermined baseline pressure value may range between 60 mmHg to 100 mmHg, although in some embodiments the predetermined baseline pressure value may range between 65 mmHg to 75 mmHg. At decision point 202, if the cuff pressure P1 is less than the predetermined baseline pressure value, then at block 204 the microprocessor 115 instructs the pressure sensor 123 to detect a first plurality of cuff pressures from the vascular cuff 108 at a respective first set of predetermined times. For example, in some embodiments the pressure sensor 123 may detect five cuff pressures from the vascular cuff 108 at subsequent predetermined times T2, T3, T4, T5 and T6 (times in milliseconds). At block 206, the microprocessor 115 then calculates the average cuff pressure P2 from the first plurality of cuff pressures taken at the respective first set of predetermined times and stores the average cuff pressure P2 in database 119. At block 208, the microprocessor 115 instructs the pressure sensor 123 to detect a second plurality of cuff pressures from the vascular cuff 108 at a respective second set of predetermined times in which the second set of predetermined times are taken after the first set of predetermined times. For example, in some embodiments the pressure sensor 123 may detect the second plurality of cuff pressures from the vascular cuff 108 at times T7, T8, T9, T10 and T11 (times in milliseconds) which are taken after the first plurality of cuff pressures at times T2, T3, T4, T5 and T6. At block 210, the microprocessor 115 calculates the average cuff pressure P3 from the second plurality of cuff pressures taken at the respective second set of predetermined times and stores the average cuff pressure P3 in database 119.

At decision point 212, the microprocessor 115 determines whether the average cuff pressure P3 subtracted from the average cuff pressure P2 (P3−P2) is less than a Boundary Value (BV) or whether the average cuff pressure P3 subtracted from the average cuff pressure P2 (P3−P2) is greater than the Boundary Value.

At decision point 212, if P3−P2 is less than Boundary Value then the microprocessor 115 determines that the vascular cuff 108 is a large-sized vascular cuff and stores that determination in the database 119 at block 214 and if P3−P2 is not less than Boundary Value then the microprocessor 115 determines that the vascular cuff 108 is a small-sized vascular cuff and stores that determination in the database 119 at block 216. At decision point 202, if the cuff pressure P1 is not less than the predetermined baseline pressure value 70 mmHg then the microprocessor 115 determines that previous determination of cuff size for the vascular cuff 108 stored in the database 119 at block 218 is used to determine that the vascular cuff 108 is either a large-sized vascular cuff or a small-sized vascular cuff.

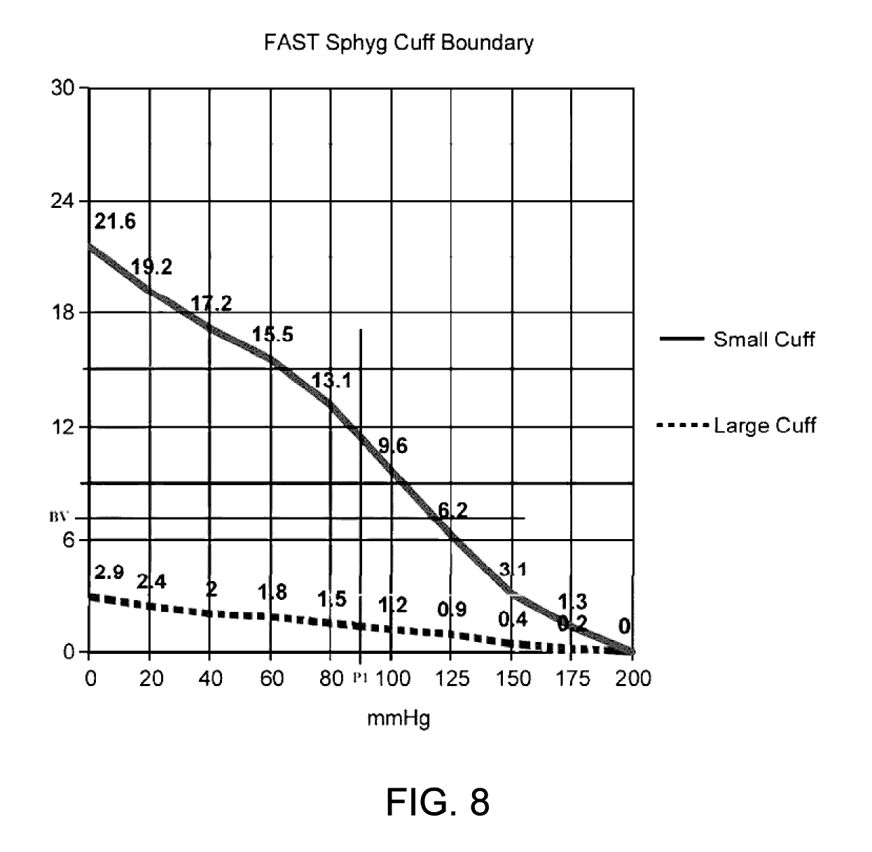

FIG. 8 shows a graph that illustrates the determination of boundary value used in the method described in FIG. 7 to determine whether the vascular cuff 108 is a small-sized vascular cuff or a large-sized vascular cuff. In one aspect, the graph plots the boundary values for a small-sized vascular cuff (shown in solid line) and a large-sized vascular cuff (shown in dashed line) at various air pressures applied to the vascular cuff 108. In particular, boundary value calculations (e.g., slope value (P3−P2)) for both the small-sized and large-sized vascular cuffs are plotted across the complete inflation range.

As shown in FIG. 8, in a series of trials that were conducted the boundary value (BV) along the Y-axis was selected through a trial of inflations using a large-sized vascular cuff in which slope value (P3−P2) was collected at a plurality of different air pressures along the X-axis ranging between 0 mmHg to 200 mmHg of air pressure. For example, if the largest value recorded is 5 based on the plurality of recorded air pressures, then add two integers above the largest recorded value to calculate BV, which in this case the BV would be 7.

As further shown in FIG. 8, in a series of trials that were conducted the cuff pressure P1 along the X-axis was selected through a trial of inflations using a small-sized vascular cuff in which the slope value (P3−P2) was collected at plurality of different air pressures in which P1 was set to fit safely behind where the slope value crosses the BV value (moving along the X-axis). Based on the foregoing, in some embodiments any slope value below 7 and less than 90 mmHg of air pressure is stored in database 119 as a large-sized vascular cuff and any slope value above 7 and less than 90 mmHg is stored in database 119 as a small-sized vascular cuff. In addition, any slope value above an air pressure of 90 mmHg the microprocessor 115 retrieves the previously stored determination for the vascular cuff 108 (large-sized vascular cuff or small-sized vascular cuff) in database 119 to determine the size of the vascular cuff 108 connected to the sphygmomanometer apparatus 102.

Once the size of the vascular cuff 108 has been determined, the microprocessor 115 adjusts the power being supplied to the rolling pump 112 by controlling the amount of power being supplied to the rolling pump 112. For example, if a large-sized vascular cuff 108 is detected by the microprocessor 115, the rolling pump 112 operates at 100% power level, while if a small-sized vascular cuff 108 is determined by the microprocessor 115 the rolling pump 112 will operate at about 35% power level, although other power levels less than 100% are contemplated when a small-sized vascular cuff 108 is used. This method of operation allows the sphygmomanometer apparatus 102 to inflate the vascular cuff 108 at a reasonable inflation speed based on the size of the vascular cuff 108 with a single actuation of the sphygmomanometer apparatus 102.

In some embodiments, the sphygmomanometer apparatus 102 may include a safety feature that automatically terminates inflation of the vascular cuff 108 if the pressure detected by the pressure sensor 123 exceeds a predetermined pressure value, for example 270 mmHg, to prevent over-inflation of the vascular cuff 108 and potential injury to the patient.

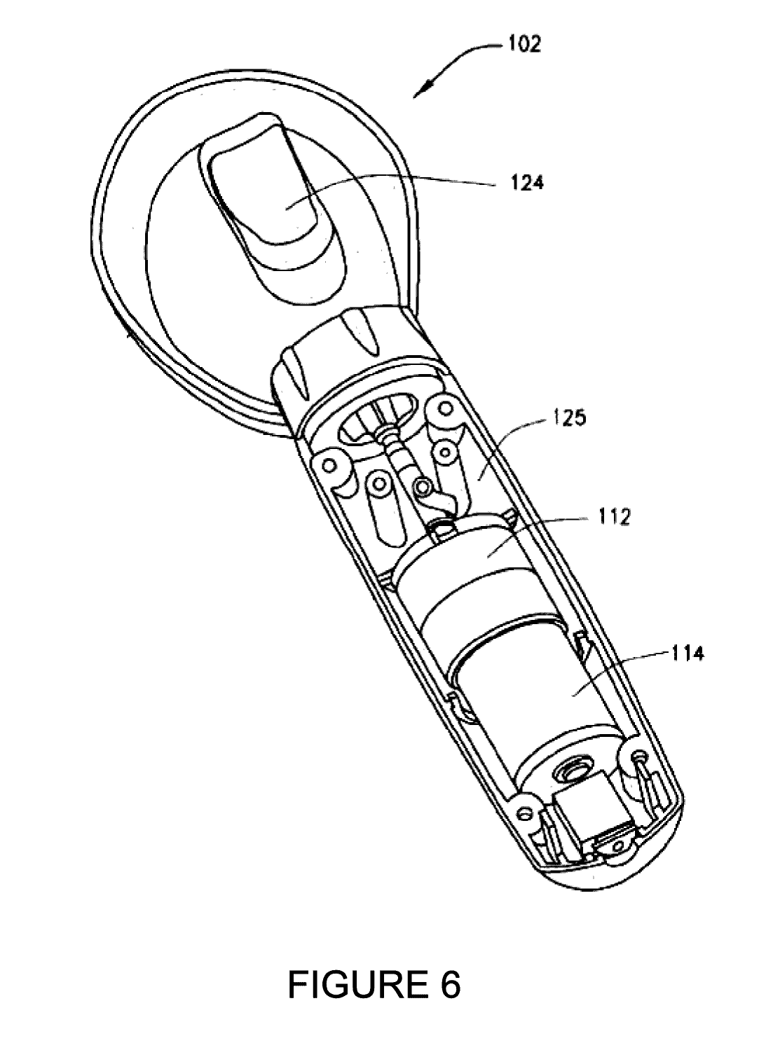

As shown in FIGS. 1 and 5, in some embodiments the sphygmomanometer apparatus 102 includes an air outlet 111 configured to engage the hollow tubing 106 (FIG. 1), which is connected to the vascular cuff 108 to allow inflation of the vascular cuff 108 by the rolling pump 112. During operation of the pump 112, air flow generated by the pump 112 flows through the hollow tubing 106 to inflate the vascular cuff 108, which is configured to be wrapped around an individual's arm (or other location along the body of an individual) when monitoring an individual's blood pressure as shall be discussed in greater detail below.

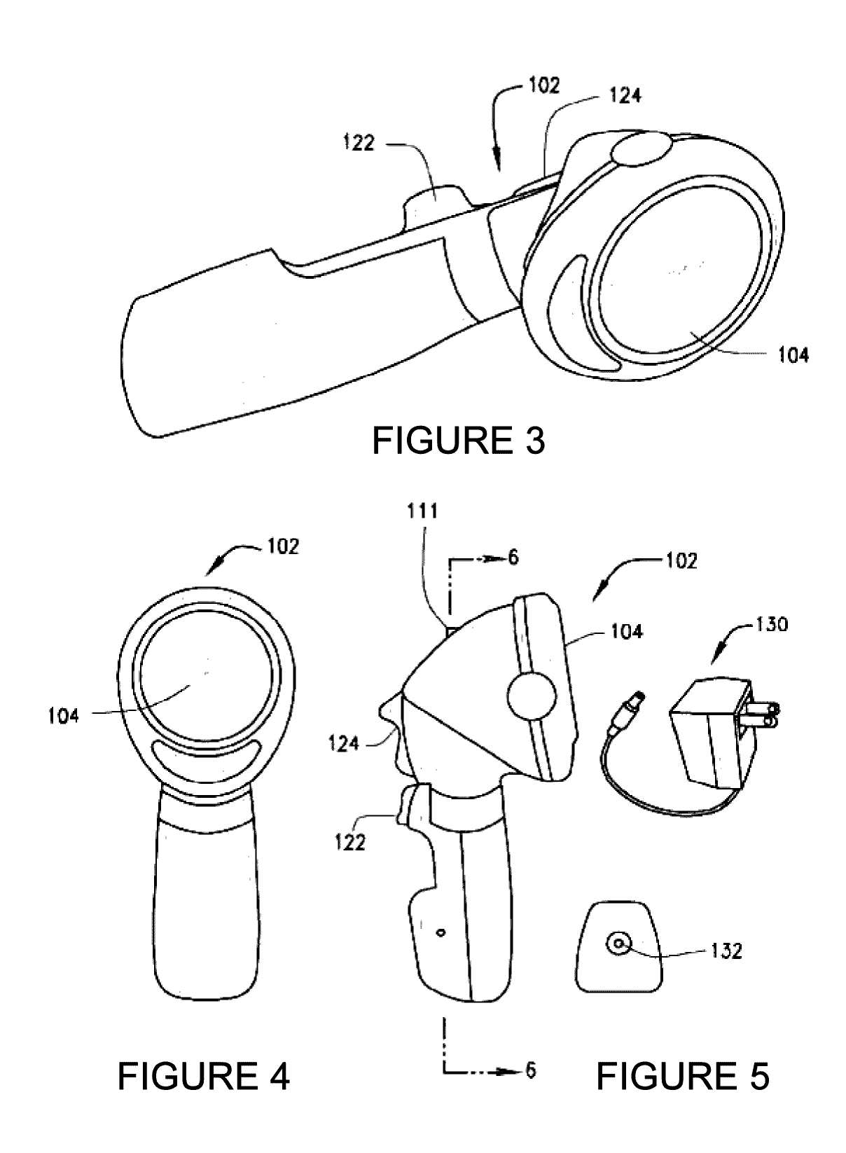

Referring to FIGS. 3-6, the sphygmomanometer apparatus 102 may further include an inflationary button 122 that activates and terminates operation of the motor 114 in order to turn on and off the operation of the rolling pump 112. In operation, the practitioner must depress and maintain the inflationary button 122 in the depressed state to maintain the inflationary button 122 in an “ON” position, which activates the rolling pump 112. Once the practitioner releases the inflationary button 122, the inflationary button 122 is placed in the “OFF” position and the operation of the rolling pump 112 ceases until the practitioner once again depresses the inflationary button 122.

As further shown, the motor 114 may include one or more batteries 118 to provide power to the motor 114 for operating the rolling pump 112. The sphygmomanometer apparatus 102 may include an internal chamber 125 configured to encase the rolling pump 112, motor 114, batteries 118, pressure sensor 123, tee connector 120 and printed circuit board 127. In some embodiments, the sphygmomanometer apparatus 102 may be connected directly to an electrical outlet (not shown) through a power adapter 130 (FIG. 5). As shown in FIG. 5, the sphygmomanometer apparatus 102 may include a DC jack 132 that receives an AC adapter 130, which is used to charge one or more batteries 118.

As further shown, the sphygmomanometer apparatus 102 may include a gauge 104 that includes a dial or digital readout for providing a visual indication of air pressure within the vascular cuff 108. The gauge 104 may be connected to a valve 116 that provides selective fluid flow communication between the pump 112, the gauge 104, and the vascular cuff 108 when actuated.

As shown in FIGS. 3 and 5, the sphygmomanometer apparatus 102 may include an air pressure release trigger 124 that allows for the deflation of the vascular cuff 108 when actuated by releasing air pressure from vascular cuff 108. Once inflation of the vascular cuff 108 is terminated by releasing the inflationary button 122, the practitioner may then release the air pressure from the vascular cuff 108 by actuating the air pressure release trigger 124 such that air pressure is released to the atmosphere. In some embodiments, the air pressure release trigger 124 may be a trigger mechanism, a button mechanism, a switch mechanism, or a dial mechanism for releasing air pressure from the sphygmomanometer apparatus 102.

In one method of use of the semi-automatic sphygmomanometer 100, the vascular cuff 108 is wrapped snugly around an individual's arm or other location along the body of the individual. For example, the vascular cuff 108 may be positioned so that the lower part of the vascular cuff 108 is approximately 1 inch above the inner bend of the individual's elbow and over the brachial artery. After inflating the vascular cuff 108 as discussed above until the gauge 104 reads a pressure of about 200 mm Hg, the practitioner may then place one end of a stethoscope (not shown) over the individual's brachial pulse and actuate the air pressure release trigger 124 to begin deflating the vascular cuff 108. As air pressure falls and the vascular cuff 108 deflates, the practitioner listens carefully for the first audible sound of the individual's heartbeat or pulse through the stethoscope. The first audible sound of the heartbeat or pulse heard through the stethoscope indicates the individual's systolic blood pressure. If applying a pressure of 200 mmHg to the vascular cuff 108 proves insufficient to eliminate audible sounds of the heartbeat or should the practitioner choose to make a second attempt at distinguishing the first audible sounds, the practitioner may prefer to again inflate the vascular cuff 108 without first reducing the pressure initially to zero. Once systolic blood pressure has been satisfactorily established, the remaining air pressure is then released from the vascular cuff 108 by actuation of the air pressure release trigger 124. In some embodiments, a Doppler device may be used, rather than a stethoscope, to listen for the heartbeat or pulse.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A semi-automatic sphygmomanometer apparatus comprising:
a motor in operative connection to a pump for generating air pressure;
an inflationary button in operative connection with the motor for activating and terminating operation of the motor;
an air pressure release trigger for releasing air pressure generated by the pump;
an air outlet in fluid flow communication with the pump;
a hollow tubing having a first end in fluid flow communication with the air outlet;
an inflatable cuff in fluid flow communication with the hollow tubing for providing air pressure from the pump to the inflatable cuff;
a pressure sensor in operative communication with the pump for detecting the degree of air pressure generated by the pump to the inflatable cuff; and
a processor in operative communication with the pressure sensor for receiving data indicative of the air pressure generated by the pump to the inflatable cuff, wherein the processor is operable to determine whether the inflatable cuff is configured to have a large volume or a small volume prior to the inflatable cuff being re-inflated based on the processor determining whether a slope value between a first average cuff pressure (P2) detected by the pressure sensor over a first set of predetermined times and a second average cuff pressure (P3) detected by the pressure sensor over a second set of predetermined times is greater than or less than a boundary value, wherein the processor determines that the inflatable cuff has a large volume if the slope value P3−P2 is less than the boundary value and wherein the processor determines that the inflatable cuff has a small volume if the slope value P3−P2 is greater than the boundary value.

2. The semi-automatic sphygmomanometer apparatus of claim 1, wherein the processor calculates a boundary value based on a slope value between P3−P2 for the inflatable cuff configured to have the large volume and P3−P2 for the inflatable cuff configured to have the small volume.

3. The semi-automatic sphygmomanometer apparatus of claim 1, wherein the boundary value comprises a value that is two whole integers greater than P3−P2 for the inflatable cuff configured to have the large volume for a particular air pressure being applied to the inflatable cuff.

4. The semi-automatic sphygmomanometer of apparatus claim 1, wherein the processor is in operative communication with a database in which a respective boundary value defined by P3−P2 is determined for a respective air pressure being applied to the inflatable cuff.

5. The semi-automatic sphygmomanometer apparatus of claim 4, wherein the respective air pressure ranges of the inflatable cuff is between 0 mmHG to 200 mmHg prior to re-inflation of the vascular cuff.

6. The semi-automatic sphygmomanometer apparatus of claim 1, wherein the boundary value calculated by the processor defines a slope defined by P3−P2 such that any boundary value below a value of 7 is determined by the processor to be the inflatable cuff configured with the large volume and any boundary value above a value of 7 is determined by the processor to be the inflatable cuff configured with the small volume.

7. The semi-automatic sphygmomanometer apparatus of claim 1, wherein the processor instructs the pump to generate an amount of air pressure within the inflatable cuff based on whether the processor determines whether the inflatable cuff is configured with a large volume or a small volume.

8. The semi-automatic sphygmomanometer apparatus of claim 1, wherein any slope value of P3−P2 for an air pressure of 90 mmHg or greater detected by the pressure sensor requires the processor to determine that the inflatable cuff is the last determined configuration of the inflatable cuff.

9. The semi-automatic sphygmomanometer apparatus of claim 1, further comprising:
a database in operative communication with the processor, the database comprising a boundary value for each respective air pressure detected by the pressure sensor for the inflatable cuff.

10. A process for determining the size of an inflatable cuff comprising:
providing sphygmomanometer apparatus comprising:
a motor in operative connection to a pump for generating air pressure;
an inflationary button in operative connection with the motor for activating and terminating operation of the motor;
an air pressure release trigger for releasing air pressure generated by the pump;
an air outlet in fluid flow communication with the pump;
a hollow tubing having a first end in fluid flow communication with the air outlet;
an inflatable cuff in fluid flow communication with the hollow tubing for providing air pressure from the pump to the inflatable cuff;
a pressure sensor in operative communication with the pump for detecting the degree of air pressure generated by the pump to the inflatable cuff; and
a processor in operative communication with the pressure sensor for receiving data indicative of the air pressure generated by the pump to the inflatable cuff;
detecting a first cuff pressure of the inflatable cuff by the pressure sensor;
determining whether the first cuff pressure by the processor is less than a predetermined baseline pressure value;
detecting a first plurality of cuff pressures by the pressure sensor at a respective first set of predetermined times and calculating by the processor a first average cuff pressure value based on the first plurality of cuff pressures if the processor determines if the processor determines that the first cuff pressure is less than the predetermined baseline value;
determining a second plurality of cuff pressures by the pressure sensor at a respective second set of predetermined times and calculating by the processor a second average cuff pressure value based on the second plurality of times;
subtracting the first average cuff pressure value from the second average cuff pressure to determine a slope value for the inflatable cuff; and
determining by the processor whether the slope value is greater than or less than a boundary value, wherein if the slope value is less than the boundary value then the processor determines that the inflatable cuff is configured with a large volume.

11. The process of claim 10, wherein if the processor calculates that the slope value is greater than the boundary value then the processor determines that the inflatable cuff is configured with a small volume.

12. The process of claim 10, further comprising:
receiving data indicative of the air pressure generated by the pump to the inflatable cuff, wherein the processor determines whether the inflatable cuff is configured to have a large volume or a small volume prior to the inflatable cuff being re-inflated based on the processor determining whether a difference between a first average cuff pressure detected by the pressure sensor over a first set of predetermined times and a second average cuff pressure (P3) detected by the pressure sensor over a second set of predetermined times is greater than or less than the boundary value.

13. The process of claim 10, wherein the first set of predetermined times occurs before the second set of predetermined times when the pressure sensor is detected the first plurality of pressures and the second plurality of pressure, respectively.

14. The process of claim 10, wherein if the first cuff pressure is greater than the predetermined baseline pressure value than the processor determines that the vascular cuff is configured to be either a large volume or small volume based on the previous determination by the processor on whether the vascular cuff is configured with a large volume or a small volume.

15. The process of claim 10, wherein the predetermined baseline pressure value is in a range between 65 mmHg to 75 mmHg.

16. The process of claim 10, wherein the predetermined baseline pressure value is in a range between 60 mmHg to 100 mmHg.

17. The process of claim 10, further comprising:
re-inflating the inflatable cuff based on the determination by the processor that the inflatable cuff is configured with a large volume or a small volume.

18. A method for determining the size of an inflatable cuff comprising:
detecting a first cuff pressure of an inflatable cuff;
determining whether the first cuff pressure is less than a predetermined baseline pressure value;
detecting a first plurality of cuff pressures at a respective first set of times and calculating a first average cuff pressure value based on the first plurality of cuff pressures if the first cuff pressure is less than the predetermined baseline value;
determining a second plurality of cuff pressures at a respective second set of predetermined times and calculating a second average cuff pressure value based on the second set of predetermined times;
subtracting the first average cuff pressure value from the second average cuff pressure to determine a slope value for the inflatable cuff; and
determining whether the slope value by the processor is greater than or less than a boundary value, wherein if the slope value is less than the boundary value then a determination is made that the inflatable cuff is configured with a large volume.

19. The method of claim 18, wherein if the slope value is greater than the boundary value then a determination is made that the inflatable cuff is configured with a small volume.

20. The method of claim 18, wherein the first set of predetermined times occur before the second set of predetermined times.
